(19) United States
(12) Patent Application Publication
(10) Pub. No.: US 2010/0050354 A1
(43) Pub. Date: Mar. 4, 2010

(54) Instrument for cleaning surgical instruments during surgery

(76) Inventor: Timothy S. Neavin, W. Hollywood, CA (US)

Correspondence Address: Samuel L. Alberstadt, 11500 San Vicente Blvd., No. 210, Los Angeles, CA, 90049, US

(21) Appl. No.: 12/380,452

(22) Filed: Feb. 26, 2009

Related U.S. Application Data
(60) Provisional application No. 61/067,517, filed on Feb. 28, 2008.

(51) Int. Cl. A46B 9/02; A47L 25/00; A61B 19/00

(52) U.S. Cl. 15/160; 15/244.4

(57) ABSTRACT

An instrument for cleaning surgical instruments during surgery includes a plurality of cleaning surfaces. The surfaces can vary in roughness and can include bristles that remain constant or vary in height or thickness or both. The surfaces can be replaced and are attached to a flexible base that conforms to the shape of a surface on which the instrument is mounted, such as an arm. The instrument can also include a sponge-like surface that can be moistened from a reservoir of fluid such as a blister pack.

### CROSS REFERENCE TO RELATED APPLICATION

This is a non-provisional application based upon a provisional patent application, Ser. No. 61/067,517, filed Feb. 28, 2008, which is incorporated by reference and for which priority is claimed under 35 U.S.C. §119(e).

### BACKGROUND OF THE INVENTION

The constraints of the modern healthcare industry require healthcare providers to operate with greater efficiency and cost effectiveness to maintain a competitive advantage. Safe and cost-effective surgery relies heavily on reducing operative time, which in turn risks the reduction of surgical efficiency.

Often during surgical cases, surgical instruments become contaminated with surgical debris. Debris consists of but is not limited to bodily fluids which can be either moist, dry, or burned to the instrument. Debris can also consist of human tissue or even suture particles or other foreign bodies used during surgery. With such contamination, instruments often fail to operate properly. These instruments are then typically passed to a surgical technician, nurse, or assistant for cleaning. This cleaning poses several problems. First, surgery takes longer to complete, making the procedure less efficient as instruments are exchanged or cleaned or both, disrupting the flow of the surgery. Second, handing sharp instruments from a surgeon to and from an operative assistant poses a risk for a puncture injury from the sharp instrument to any person involved in the exchange of the sharp instrument. Puncture wounds and cuts from sharp instruments continue to be a serious problem in all facets of healthcare. Third, the passing of instruments from person to person in the surgical environment creates the potential for dropped instruments from the sterile surgical field. Dropped instruments must be replaced, delaying operative time as replacement instruments must be located, sterilized, and delivered to the operative suite.

### SUMMARY OF THE INVENTION

This invention is a apparatus that functions as a convenient, compact, sterile instrument cleaner in the sterile operative field. The apparatus is used to clean surgical instruments quickly, as surgical instruments often become less effective or function poorly as bodily fluids, tissue, and suture debris contaminate the instruments. The invention is convenient to the surgeon, surgical assistant, and surgical technician, because it reduces operative time by avoiding passing of instruments for an assistant or technician to clean. The invention also reduces sharp instrument injuries and dropped instruments by providing an immediately accessible, local cleaning surface for surgical instruments. This surface allows the user to avoid the passing of instruments to or handling of sharp instruments by a technician or assistant for cleaning. These and other advantages and purposes of the invention will become apparent from the description of the invention.

The invention includes one or more cleaning surfaces divided into three or more regions on a single pad. The backside of the pad has an adhesive backing and can be attached to most surfaces on the operative field including but not limited to the surgeon's gown, the patient, or the surgical drapes. It can also be attached directly to the surgeon, such as at his wrist. Three different cleaning surfaces are preferred. The device can consist of one or more of these surfaces as needed. A surface comprised of bristles can aid in removal of dried debris with simple swipes of the instrument through its bristles. The moist sponge surface can aid in removing small debris when the instrument is passed along its surface. A coarse or rough surface can aid in cleaning more stubborn surgical debris from instruments, such as electrocautery tips to which burned tissue and fluids may adhere.

### BRIEF DESCRIPTION OF THE DRAWINGS

In the detailed description which follows, reference will be made to the drawing following figures.

FIG. 1 is a plan view showing three surfaces of the instrument cleaner for a sterile operative field.

FIG. 2 depicts a side view of the invention.

FIG. 3 depicts a front view of the invention.

### DESCRIPTION OF THE PREFERRED EMBODIMENTS

A surgical instrument cleaner 10 consists of one or more cleaning surfaces divided into three regions on a single pad. The backside of the pad is pliable, has an adhesive backing, and it can be attached to most surfaces on the operative field including but not limited to the surgeon's gown, patient, or surgical drapes. Velcro and other conventional attachment mechanisms, such as clasps, buttons, clips, or pins, may also be used. The device can consist of one or more of three possible cleaning surfaces as needed. This permits the cleaning of a variety of instruments, such as needle drivers, forceps, scalpels, and cauterizing tips.

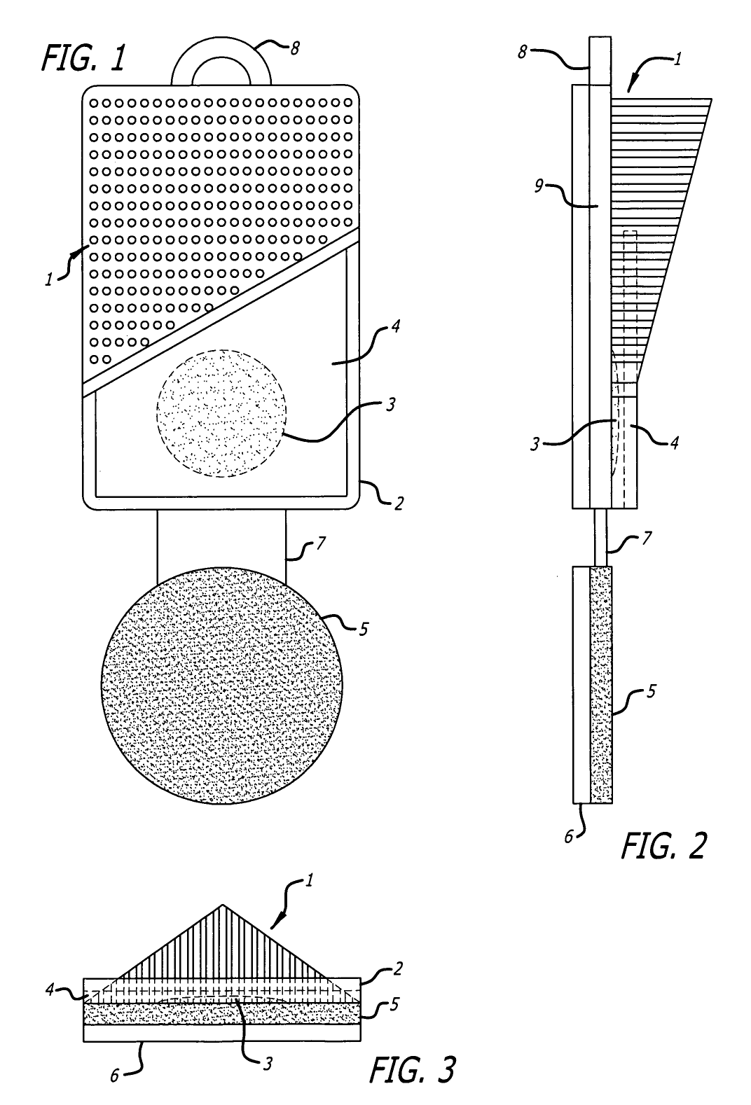

FIG. 1 depicts 3 surfaces, including a bristled surface 1, a soft, sponge-like surface 4, and a coarse surface 5. The bristles on the bristled surface may be larger on the outside and become thinner, and more pliable, toward the middle. The bristled surface can aid in removal of dried debris with simple swipes of the instrument through its bristles. The bristled surface sits adjacent to the soft, sponge-like surface 4 and is divided by a wall 2, which runs along the perimeter of the sponge-like surface 4. The soft, sponge-like surface 4 can aid in removing small debris with instrument passes along its surface, particularly when the surface is moistened by opening or puncturing the blister pack of liquid 3. Sitting either inside or just below sponge-like surface 4 is reservoir 3 that can easily be opened or punctured to allow liquid contents to empty and soak the sponge 4. One preferred embodiment of the reservoir is a blister pack. The contents of reservoir 3 may contain a disinfectant, a bactericidal agent, a lubricant, or any other liquid. A pliable bridge 7 divides the coarse surface 5 from sponge-like surface 4. The coarse surface 5 has an abrasive or rough surface. The coarse or rough surface can aid difficult-to-clean surgical debris from instruments, such as electrocautery tips to which burned tissue and body fluids may adhere.

Underlying surfaces 1, 4, and 5 is a pliable, adhesive surface 6 that allows this apparatus to stick on virtually any dry surface, including but not limited to surgical gowns, surgical drapes, or the patient. A looped hole 8 is attached to the end of the apparatus and can serve as ring to attach a pin or clip to secure the apparatus on surgical drapes, surgical gowns, or other surgical paraphernalia. The looped hole 8 is attached to a base 9 that supports 1, 2, 3, and 4. The base 9 is preferably pliable and can conform to the object on which it is placed, such as the curve of the arm of a user. Also, it is preferable that the base be comprised of a stretchable material such as elastic.

FIG. 2 depicts a side view of the invention. The bristles 1 may start off taller and become shorter as they get closer to the divider 2. The bristles may also be thicker and less pliable when they are taller, and thus, farther from divider 2. In one embodiment of the invention at least one of the surfaces could be removable and replaceable by the same or a different type of surface. Similarly, other aspects of the invention would also preferably be replaceable, such as the reservoir or blister pack 3. Those of ordinary skill in the art will understand that the surgical instrument cleaner will be used in a sterile environment. Therefore, it should be constructed of materials that can be sterilized. Different commercial embodiments are possible. For example, the surgical instrument cleaner can be constructed for single-use sterilization and packaging, so that it can be disposed of after surgery. Alternatively, as medical costs rise, hospitals and other medical facilities may prefer cleaners that are constructed in a manner that permits them to be re-sterilized and reused.

FIG. 3 is a front view of the invention and illustrates that the bristles may form a pyramid whereby the bristles are tallest in the center.

Variations of the described combinations are possible within the spirit and scope of the invention, which is to be limited only by the following claims and their equivalents.

What is claimed is:

1) A surgical instrument cleaner attachable to an individual participating in surgery, comprising:
a base;
a plurality of surfaces disposed over the base, the surfaces comprising a plurality of means for cleaning surgical debris from surgical instruments during surgery; and,
means for attaching the surgical instrument cleaner to an individual comprising one of a button, Velcro, adhesive, a clasp, a snap, a clip, or a pin.

2) The invention of claim 1, wherein the base is flexible.

3) The invention of claim 2, wherein one of the surfaces comprises bristles configured for cleaning dried debris.

4) The invention of claim 2, wherein one of the surfaces comprises bristles configured for cleaning soiled debris.

5) The invention of claim 2, further comprising a moist and spongy surface for cleaning dried debris.

6) The invention of claim 5, further comprising a container for holding a fluid for moistening the moist and spongy surface.

7) The invention of claim 2, further comprising a rough surface for cleaning difficult-to-clean debris.

8) The invention of claim 2, further comprising a divider between the cleaning surfaces.

9) The invention of claim 2, wherein at least one of the plurality of surfaces is removably fixed.
